(19) United States
(12) Patent Application Publication
(10) Pub. No.: US 2022/0369792 A1
(43) Pub. Date: Nov. 24, 2022

(54) COSMETIC DEVICE AND COSMETIC DEVICE SYSTEM

(71) Applicant: LG HOUSEHOLD & HEALTH CARE LTD., Seoul (KR)

(72) Inventors: Hyung Kyu KIM, Seoul (KR); Sung Taek OH, Seoul (KR); Jung Yong LEE, Seoul (KR); Kyung Won KIM, Seoul (KR)

(73) Assignee: LG HOUSEHOLD & HEALTH CARE LTD., Seoul (KR)

(21) Appl. No.: 17/775,410

(22) PCT Filed: Dec. 1, 2020
(86) PCT No.: PCT/KR2020/017352

§ 371 Date: May 9, 2022

(30) Foreign Application Priority Data
Feb. 24, 2020 (KR) 10-2020-0022187

(51) Int. Cl. A45D 44/00; A61B 5/00; A61H 23/02; A61N 5/067; A61N 1/44; A61N 1/32; A61H 11/00; A61N 5/06

(52) U.S. Cl. CPC A45D 44/00; A61B 5/4836; A61H 23/02; A61N 5/067; A61N 1/44; A61N 1/328; A61H 11/00; A61N 5/0616; A45D 2044/007; A61N 2005/0651; A61H 2205/022; A61H 2201/5043; A61H 2201/5007

(57) ABSTRACT

A cosmetic device according to an embodiment of the present disclosure comprises: a main body; a head module that is detachable from the main body; a control unit for recognizing the head module mounted to the main body; and a communication unit for transmitting information about the recognized head module to a mobile terminal. The control unit can control the operation of the head module mounted to the main body differently according to the information received from the mobile terminal.

### TECHNICAL FIELD

The present disclosure relates to a cosmetic device and a cosmetic device system.

### BACKGROUND ART

As people's interest in beauty increases, not only cosmetics, makeup tools, etc. are diversified, but also the types of cosmetic devices are also gradually increasing. In particular, in the past, in order for people to use a cosmetic device, there was the hassle of having to visit a hospital with a dermatologist or a beauty shop. However, recently, as portable or relatively small cosmetic devices has been released, an environment in which people can easily use cosmetic devices at home is being created.

On the other hand, since cosmetic devices are manufactured differently depending on their uses, there is an inconvenience that people have to purchase cosmetic devices for each use. For example, people have to separately purchase a cosmetic device for scalp care, a cosmetic device for skin care, a cosmetic device for wrinkle improvement, etc., which increases the purchase cost, requires a large space for storage, and may experience inconveniences that are cumbersome to manage.

### DISCLOSURE

### Technical Problem

The present disclosure is directed to providing a cosmetic device and a cosmetic device system capable of diagnosis and various care with one device.

The present disclosure is directed to providing a cosmetic device and a cosmetic device system for guiding a user to a function that may be provided according to a mounted head module.

The present disclosure is directed to providing a cosmetic device and a cosmetic device system in which a state of the cosmetic device may be checked through a mobile terminal.

The present disclosure is directed to providing a cosmetic device and a cosmetic device system capable of both diagnosing and caring for a user's condition.

The present disclosure is directed to providing a cosmetic device and a cosmetic device system that provide various care functions based on a diagnosis result.

The present disclosure is directed to providing a cosmetic device and a cosmetic device system capable of providing an ampoule.

### Technical Solution

A cosmetic device according to an embodiment of the present disclosure includes a main body, a head module detachable from the main body, a control unit for recognizing a head module mounted on the main body, and a communication unit for transmitting information on the recognized head module to a mobile terminal and the control unit may differently control an operation of the head module mounted on the main body according to information received by the mobile terminal.

The control unit may differently control the operation of the head module according to information received from the mobile terminal even when the head module mounted on the main body is the same.

When a care module is mounted after a diagnosis module is mounted on the main body, the control unit may control the care module based on a diagnosis result of the diagnosis module.

The head module may include at least one of a diagnosis module, a skin care module, a scalp care module, and an ampoule care module.

Each of the diagnosis module, the skin care module, the scalp care module, and the ampoule care module may include a resistance element having a different resistance value, and the control unit may calculate the resistance value of the resistance element provided in the head module mounted on the main body to recognize the head module.

A space in which a cartridge is mounted and a hole through which an ampoule accommodated in the cartridge passes may be formed in the ampoule care module.

A cosmetic device system according to an embodiment of the present disclosure may include a cosmetic device with a detachable head module, and a mobile terminal that determines a method of operating the cosmetic device based on information on the head module mounted on the cosmetic device and transmits information on the determined operation method to the cosmetic device, wherein the cosmetic device may differently control an operation of the head module according to information received by the mobile terminal.

The cosmetic device system may differently control the operation of the head module according to information received from the mobile terminal even when the mounted head module is the same.

The mobile terminal may display an operation mode provided by the head module based on the information on the head module.

The mobile terminal may differently display the operation mode when the information on the head module is different.

When the mobile terminal receives the information on a diagnostic module and then receives information on a care module, the mobile terminal may display an icon that indicates an operation mode provided by the care module and an operation mode recommended according to a diagnosis result of the diagnostic module among the operation modes.

When the mobile terminal receives the information on the diagnosis module and then receives the information on the care module, the mobile terminal may generate an operation mode recommended based on a function provided by the care module and the diagnosis result of the diagnosis module.

When the mobile terminal receives the information on the diagnostic module and then receives the information on the care module, the mobile terminal may determine an operation method so that a plurality of operation modes provided by the care module are continuously executed based on the diagnostic result of the diagnostic module.

The mobile terminal may receive a user input for setting a method of operating the head module, and may generate an operation mode corresponding to the user input.

The mobile terminal may display different operating methods of the settable head module according to the head module mounted on the cosmetic device.

### Advantageous Effects

According to an embodiment of the present disclosure, there is an advantage that a diagnosis function and various care functions can be provided with only one cosmetic device.

According to an embodiment of the present disclosure, it is possible to guide a user to an operation mode that can be operated according to a head module mounted on a cosmetic device, and there is an advantage that the user can easily select an operation method of the head module.

According to an embodiment of the present disclosure, a cosmetic device has an advantage that it is possible to provide appropriate care to a user by recommending an operation mode according to a diagnosis result.

According to an embodiment of the present disclosure, in a cosmetic device to which various types of head modules can be mounted, since an operation method is determined differently depending on the head module even though a user does not separately input the type of each head module, there are advantages that an operation error can be minimized.

### DESCRIPTION OF DRAWINGS

FIG. 1 is a block diagram of a cosmetic device system according to an embodiment of the present disclosure.

FIG. 2 is a control block diagram of a cosmetic device according to an embodiment of the present disclosure.

FIG. 3 is a control block diagram of a mobile terminal according to an embodiment of the present disclosure.

FIG. 4 is a perspective view illustrating a cosmetic device according to an embodiment of the present disclosure.

FIG. 5 is a perspective view illustrating a main body of the cosmetic device shown in FIG. 4.

FIG. 6 is a view illustrating a state in which a mounting part shown in FIG. 5 is viewed from a X direction.

FIG. 7 is a perspective view of a diagnostic module according to an embodiment of the present disclosure.

FIG. 8 is a perspective view of a skin care module according to an embodiment of the present disclosure.

FIG. 9 is a perspective view of a scalp care module according to an embodiment of the present disclosure.

FIG. 10 is a perspective view of an ampoule care module according to an embodiment of the present disclosure.

FIG. 11 is a cross-sectional view of the ampoule care module shown in FIG. 10.

FIG. 12 is a flowchart illustrating a method of operating a cosmetic device system according to an embodiment of the present disclosure.

FIG. 13 is a view illustrating a first example of a screen displayed by a mobile terminal to determine a method of operating a cosmetic device according to an embodiment of the present disclosure.

FIG. 14 is a view illustrating a second example of the screen displayed by the mobile terminal to determine the method of operating the cosmetic device according to the embodiment of the present disclosure.

FIGS. 15 and 16 are views illustrating a third example of the screen displayed by the mobile terminal to determine the method of operating the cosmetic device according to the embodiment of the present disclosure.

### MODES OF THE INVENTION

Hereinafter, embodiments disclosed in the present specification will be described in detail with reference to the accompanying drawings, however, the same elements are designated by the same reference numerals, and repeated description thereof will be omitted.

Suffixes “module” and “part” for elements used in the following descriptions are given or used just for convenience in writing the specification, and do not have meanings or roles distinguishable between them.

In addition, in describing embodiments of the present disclosure, when detailed description of a known function is deemed to unnecessarily blur the gist of the present disclosure, the detailed description will be omitted. Further, accompanying drawings are only for easily understanding embodiments disclosed in the present disclosure, and the technical spirit disclosed in the present disclosure are not limited by the accompanying drawings, and it should be understood that the present invention includes all modifications, equivalents, and alternatives falling within the spirit and scope of the claims.

The terms including an ordinal number such as first, second, and the like may be used to describe various elements, but the elements are not limited by the terms. The terms are only used to distinguish one element from another.

The singular expression includes the plural expression unless the context clearly dictates otherwise.

It should be understood that the terms such as “comprises”, “have”, or the like, when used in the present application, specify the presence of stated features, integers, steps, operations, elements, and/or components, but do not preclude the presence or addition of one or more other features, integers, steps, operations, elements, components, and/or groups thereof.

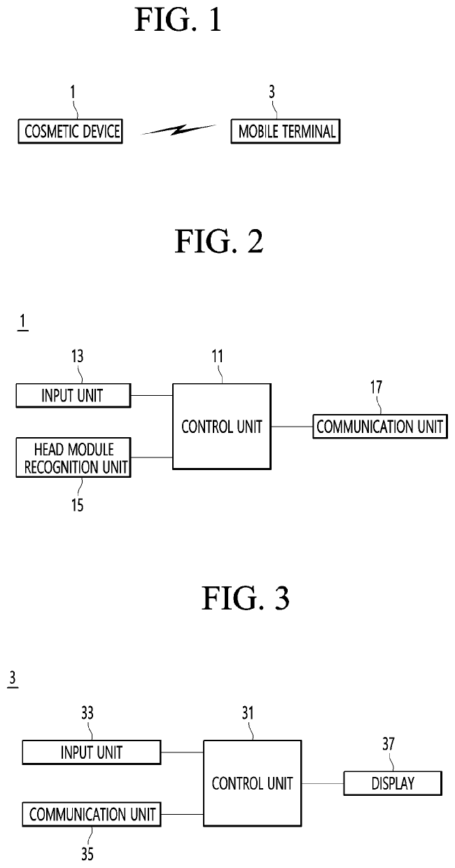

FIG. 1 is a block diagram of a cosmetic device system according to an embodiment of the present disclosure.

The cosmetic device system according to an embodiment of the present disclosure may include a cosmetic device 1 and a mobile terminal 3. The cosmetic device 1 and the mobile terminal 3 may transmit and receive signals to and from each other.

The cosmetic device 1 may be a device that diagnoses a user's condition or provides a care function.

The cosmetic device 1 may have a head replaceable structure in which various head modules may be mounted and transmit information about the mounted head module to the mobile terminal 3.

The mobile terminal 3 may display information about the cosmetic device 1. For example, when the mobile terminal 3 receives information about the head module from the cosmetic device 1, the mobile terminal 3 may display operation modes currently available to the cosmetic device 1 based on the received information.

The user may determine a method of operating the cosmetic device 1 through the mobile terminal 3. The mobile terminal 3 may transmit a control signal to the cosmetic device 1 so that the cosmetic device 1 operates according to the determined operation method.

The mobile terminal 3 may be a portable mobile terminal. For example, the mobile terminal 3 may include a mobile phone such as a smart phone, a wearable device such as a smart watch, and a computer such as a laptop computer, a tablet, and the like, but the present disclosure is not limited thereto.

FIG. 2 is a control block diagram of a cosmetic device according to an embodiment of the present disclosure.

The cosmetic device 1 may include at least some or all of a control unit 11, an input unit 13, a head module recognition unit 15, and a communication unit 17.

The control unit 11 may control the overall operation of the cosmetic device 1. The control unit 11 may control each of the input unit 13, the head module recognition unit 15, and the communication unit 17.

The input unit 13 may receive an input for controlling an operation of the cosmetic device 1. The input unit 13 may be implemented in a form of a physical key button or a touch pad capable of receiving a touch input.

For example, the input unit 13 may receive an input for controlling the power of the cosmetic device 1 to be turned on/off, but this is merely an example, and the input unit 13 may receive various inputs for controlling the operation of the cosmetic device 1.

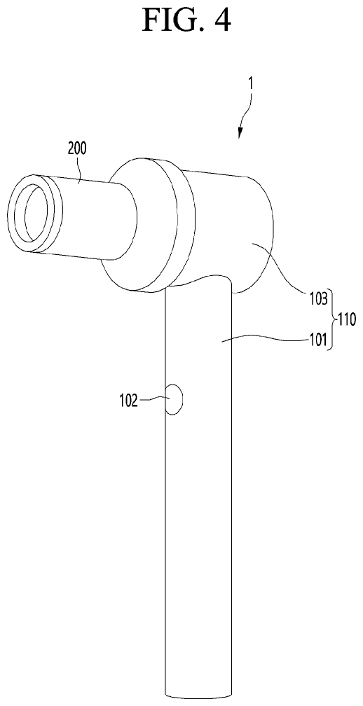

The head module recognition unit 15 may recognize a head module 200 (see FIG. 4) mounted on the cosmetic device 1. The head module recognition unit 15 may not only recognize whether the head module 200 (see FIG. 4) is mounted, but also recognize the type of the head module 200 (see FIG. 4). That is, the head module recognition unit 15 may recognize whether the head module 200 (see FIG. 4) is mounted on a main body 110 (see FIG. 4) and the type of the head module 200 (see FIG. 4) when the head module 200 (see FIG. 4) is mounted on the main body 110 (see FIG. 4). For example, the head module 200 (see FIG. 4) may include a resistance element having a different resistance value depending on the type thereof, and the head module recognition unit 15 may calculate a resistance value of the mounted head module 200 (see FIG. 4) to recognize the type of the head module.

The communication unit 17 may communicate with an external device, and the external device may be the mobile terminal 3, but the present disclosure is not limited thereto.

The communication unit 17 may include a communication module such as a Bluetooth module and a Wi-Fi module.

Meanwhile, in FIG. 2, only the control unit 11, the input unit 13, the head module recognition unit 15, and the communication unit 17 are shown as the configuration of the cosmetic device 1, but this is merely an example for convenience of description. Some of the components shown in FIG. 2 may be omitted, or other components may be further added.

FIG. 3 is a control block diagram of a mobile terminal according to an embodiment of the present disclosure.

The mobile terminal 3 may include at least some or all of a control unit 31, an input unit 33, a communication unit 35, and a display unit 37.

The control unit 31 may control an operation of the mobile terminal 3. The control unit 31 may control each of the input unit 33, the communication unit 35, and the display unit 37.

The input unit 33 may receive an input for controlling the operation of the mobile terminal 3. The input unit 33 may be implemented in a form of a physical key button or a touch pad capable of receiving a touch input.

The communication unit 35 may communicate with an external device, and the external device may be the cosmetic device 1, but the present disclosure is not limited thereto.

The communication unit 17 may include a communication module such as a Bluetooth module, a Wi-Fi module, and a mobile communication module.

The display unit 37 may display various information. The display unit 37 may display information about the cosmetic device 1.

Meanwhile, in FIG. 3, only the control unit 31, the input unit 33, the communication unit 35, and the display unit 37 are shown as the configuration of the mobile terminal 3, but this is merely an example for convenience of description, some of the components shown in FIG. 3 may be omitted, or other components may be further added.

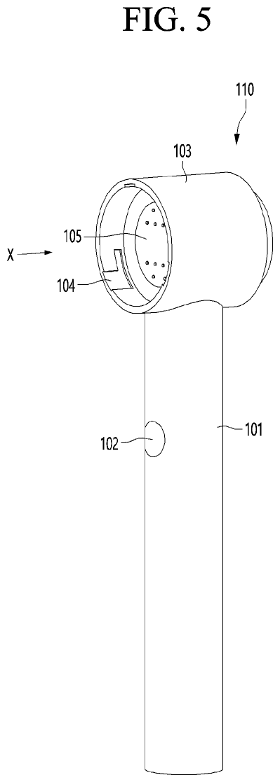

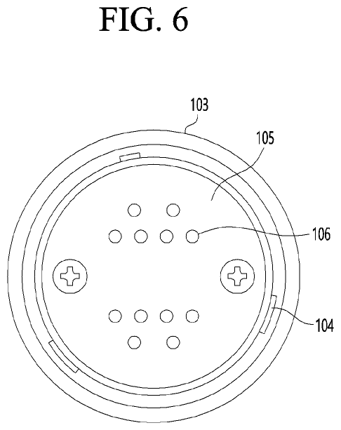

FIG. 4 is a perspective view illustrating a cosmetic device according to an embodiment of the present disclosure, FIG. 5 is a perspective view illustrating a main body of the cosmetic device shown in FIG. 4, and FIG. 6 is a view illustrating a state in which a mounting part shown in FIG. 5 is viewed from a X direction.

The cosmetic device 1 may include the main body 110 and the head module 200 detachable from the main body 110. The head module 200 may be detachable from the cosmetic device 1.

The main body 110 may include a supporter 101 and a mounting part 103, and the head module 200 may be attached to and detached from the mounting part 103. That is, the head module 200 may be mounted on or separated from the mounting part 103.

The supporter 101 may be disposed under the mounting part 103.

The supporter 101 may have a cylindrical shape. The supporter 101 may be a handle of the cosmetic device 1.

At least one button 102 may be formed on the supporter 101, and the button 102 may be one configuration of the input unit 13. The input unit 13 may receive a user input through the button 102. For example, the button 102 may be a power button for turning on/off the power of the cosmetic device 1, but this is merely illustrative.

The inside of the supporter 101 and the inside of the mounting part 103 may communicate with each other, and accordingly, a configuration disposed inside the supporter 101 and a configuration disposed inside the mounting part 103 may be connected through a wire or the like.

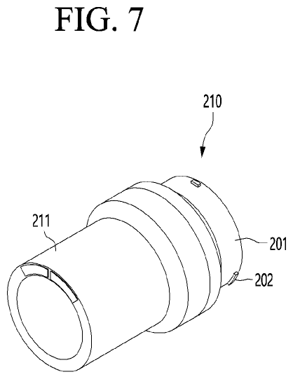

The mounting part 103 may be disposed on the supporter 101. The head module 200 may be attached to and detached from the mounting part 103. The mounting part 103 may include an accommodating part 104 in which a part of the head module 200 is accommodated. For example, the head module 200 may be mounted on the mounting part 103 while rotating in a clockwise or counterclockwise direction in a state in which a part of the head module 200 is fitted inside the mounting part 103. At this time, the protrusion 202 (see FIGS. 7 to 10) formed on the head module 200 may be accommodated in the accommodating part 104, and thus the head module 200 may be fixed to the mounting part 103.

In addition, when the head module 200 is mounted on the mounting part 103, at least a part of the head module 200 may be in contact with a substrate 105. In particular, when the head module 200 is mounted on the mounting part 103, a resistance element (not shown) of the head module 200 may be in contact with a contact pin 106 of the substrate 105.

The substrate 105 may be connected to the head module recognition unit 105. At least one contact pin 106 for recognizing resistance may be formed on the substrate 105. The head module recognition unit 105 may calculate a resistance value of the resistance element recognized through the contact pin 106.

Each of the control unit 11, the input unit 13, the head module recognition unit 15, and the communication unit 17 may be disposed in the supporter 101 or the mounting part 103. For example, the control unit 11 and the head module recognition unit 15 may be disposed inside the mounting part 103, and the input unit 13 and the communication unit 17 may be disposed inside the supporter 101, but this is merely illustrative, the present disclosure is not limited thereto.

In addition, since a shape and arrangement of the supporter 101 and the mounting part 103 shown in FIGS. 4 and 5 are merely illustrative, the present disclosure is not limited thereto.

Meanwhile, the type of the head module 200 capable of mounting on the mounting part 103 may be various. The head module 200 may include a diagnosis module, a care module, and the like, and the care module may include a skin care module, a scalp care module, an ampoule care module, and the like. In addition, the skin care module may include a first skin care module (general skin care module) that provides care to the skin of the entire face, a second skin care module (local area skin care module) that provides care only to a predetermined area of the face, or the like. However, since the above-described head modules are merely examples for convenience of description, the present disclosure is not limited thereto. For example, the head module 200 may include a roller massage module or the like.

In addition, the head module 200 may include resistance elements having different resistance values depending on the type thereof. For example, each of the diagnostic module, the first skin care module, the second skin care module, the scalp care module, and the ampoule care module may include resistance elements having different resistance values.

Next, a type of a head module according to an embodiment of the present disclosure will be described with reference to FIGS. 7 to 11.

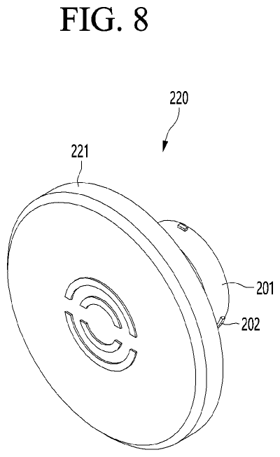

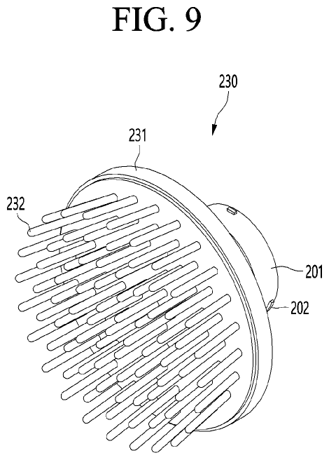

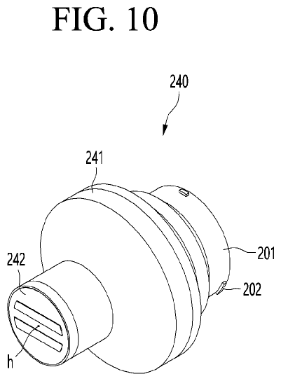

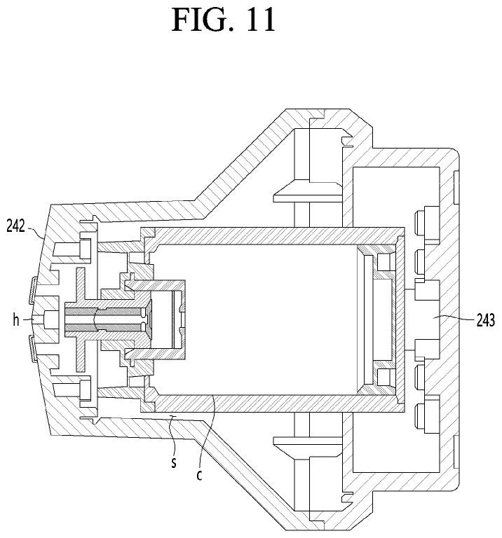

FIG. 7 is a perspective view of a diagnostic module according to an embodiment of the present disclosure, FIG. 8 is a perspective view of a skin care module according to an embodiment of the present disclosure, FIG. 9 is a perspective view of a scalp care module according to an embodiment of the present disclosure, FIG. 10 is a perspective view of an ampoule care module according to an embodiment of the present disclosure, and FIG. 11 is a cross-sectional view of the ampoule care module shown in FIG. 10.

The diagnosis module 210 shown in FIG. 7 may be a kind of the head module 200. The diagnosis module 210 may include a connector 201 and a diagnosis part 211. The connector 201 may be connected to the mounting part 103. The connector 201 may have a protrusion 202 to be fixed to the mounting part 103. The protrusion 202 may be formed on an outer surface of the connector 201.

The diagnosis unit 211 may be in contact with skin, scalp, or the like. The diagnosis part 211 may include a camera (not shown), an oil sensor (not shown), a moisture sensor (not shown), an LED (not shown), and the like. The control unit 11 may diagnose a condition of the skin, scalp, etc. in contact with the diagnosis part 211 through the camera (not shown), the oil sensor (not shown), the moisture sensor (not shown), the LED (not shown), or the like.

A skin care module 220 shown in FIG. 8 may be a kind of the head module 200. The skin care module 220 may include a connector 201 and a care part 221. The connector 201 may be connected to a mounting part 103. The connector 201 may have a protrusion 202 to be fixed to the mounting part 103. The protrusion 202 may be formed on an outer surface of the connector 201.

The care part 221 may include an LED (not shown), a high-frequency laser (not shown), an ion generating part (not shown), a vibration generating part (not shown), a galvanic part (not shown), a cooling part (not shown), a heating part (not shown), and the like. The control unit 11 may operate at least one of an LED (not shown), a high-frequency laser (not shown), an ion generating part (not shown), a vibration generating part (not shown), a galvanic part (not shown), a cooling part (not shown), and a heating part (not shown) to provide a care function to the skin in contact with the care part 221.

The skin care module 220 may provide the care function to any areas of the face. Meanwhile, according to the embodiment, the skin care module may be manufactured to fit a specific part such as eye rims or a mouth area, and in this case, the shape may be different from that shown in FIG. 8. That is, there may be a separate skin care module that provides a special care function suitable for a specific area based on characteristics of each specific area, such as the eye rims, where wrinkles are more likely to occur than other areas. For example, when the skin care module applicable to the entire face is the first skin care module, there may be the second skin care module for providing a special care function to the eye area and the third skin care module for providing a special care function to the mouth area, etc. may exist separately. In this case, shapes of the first skin care module, the second skin care module, and the third skin care module may be different from each other.

A scalp care module 230 shown in FIG. 9 may be a kind of the head module 200. The scalp care module 230 may include a connector 201 and a care part 231. The connector 201 may be connected to the mounting part 103. The connector 201 may have a protrusion 202 to be fixed to the mounting part 103. The protrusion 202 may be formed on an outer surface of the connector 201.

A plurality of comb teeth 232 may be formed in the care part 231. In addition, the care part 231 may include the vibration generator (not shown), the LED (not shown), the ion generator (not shown), and the like.

According to the embodiment, an ion generator (not shown) may be formed on at least a part of the plurality of comb teeth 232. In particular, the ion generator (not shown) may be disposed at an end of at least a part of the plurality of comb teeth 232. In this case, ions may be generated from the comb teeth 232, and accordingly, the ions generated from the ion generator (not shown) may be better transferred to the user's scalp or hair, and thus there is an advantage that an effect of preventing static electricity may be improved.

An ampoule care module 240 shown in FIG. 10 may be a kind of the head module 200. The ampoule care module 240 may include a connector 201 and a care part 241. The connector 201 may be connected to the mounting part 103. The connector 201 may have a protrusion 202 to be fixed to the mounting part 103. The protrusion 202 may be formed on an outer surface of the connector 201.

All the connector 201 of the diagnostic module 210, the connector 201 of the skin care module 220, the connector 201 of the scalp care module 230, and the connector 201 of the ampoule care module 240 may all have the same size and shape.

The care part 241 may apply an ampoule to a user's skin, scalp, or the like.

A space S in which a cartridge c is mounted and a hole h through which the ampoule accommodated in the cartridge c passes may be formed in the care part 241. In addition, a motor 243 for discharging the ampoule accommodated in the cartridge c to the outside of the cartridge c may be disposed inside the care part 241. The motor 243 may include an airless pump.

The cartridge c may accommodate the ampoule therein and may be attached to and detached from the inside of the care part 241. That is, the cartridge c may have a form that may be replaced in the care part 241.

The hole h may be a hole formed so that the ampoule of the cartridge c mounted inside the care part 241 is applied to the skin, scalp, or the like. A contact surface 242 in contact with the user's skin, scalp, or the like may be formed in the care part 241, and at least one hole h may be formed in the contact surface 242. Therefore, the ampoule discharged from the cartridge c by the motor 243 may pass through the hole h to be applied to the user's skin, scalp, or the like.

The ampoule may be used for both skin and scalp, and an ampoule for skin and an ampoule for scalp may exist separately.

Therefore, according to an embodiment, unlike FIG. 11, a cartridge accommodating the ampoule used for the skin and a cartridge accommodating the ampoule used for the scalp may be respectively mounted in the care part 241. In this case, a hole through which the ampoule used for skin passes and a hole through which the ampoule used for the scalp passes may be separately formed on the contact surface 242 of the care part 241.

In addition, the cartridge accommodating the ampoule used for the scalp and a cartridge accommodating an ampoule used for the hair may be respectively mounted in the care part 241. In this case, the care part 241 may further include a sensor (not shown) that distinguishes the scalp from the hair. For example, the sensor (not shown) may recognize a vibration pattern and recognize the scalp and the hair according to the recognized vibration pattern. The care part 241 may discharge the ampoule for the scalp or the ampoule for the hair according to a position recognized by the sensor (not shown). For example, when the sensor (not shown) recognizes that the hole h is located on the scalp, the care part 241 may discharge the ampoule for the scalp, and when the sensor (not shown) recognizes that the hole h is located on the hair, the care part 241 may discharge the ampoule for the hair.

The cartridge c may be mounted on the care part 241, and the care part 241 may include a galvanic part (not shown), a heating part (not shown), a vibration generating part (not shown), and the like as well as a cartridge c. Therefore, the control unit 11 may drive at least one of the galvanic part (not shown), the heating part (not shown), or the vibration generating part (not shown) while applying the ampoule accommodated in the cartridge c.

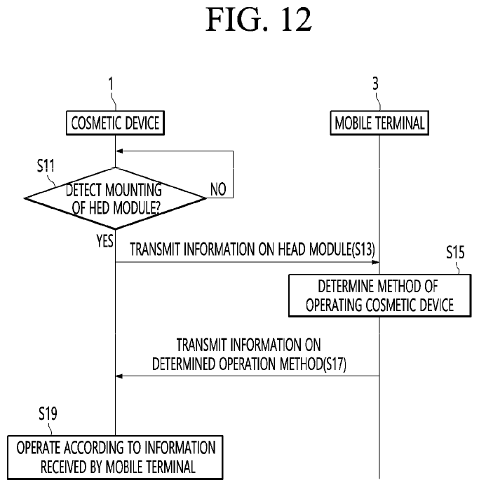

Next, FIG. 12 is a flowchart illustrating a method of operating a cosmetic device system according to an embodiment of the present disclosure.

The cosmetic device 1 may detect whether the head module 200 is mounted (S11).

The control unit 11 may recognize the head module 200 mounted on the main body 110.

The control unit 11 may recognize whether the head module 200 is mounted on the main body 110 through the head module recognition unit 15. For example, the head module recognition unit 15 may detect whether the resistance element is in contact with the contact pin 106 of the substrate 105. When it is detected that the resistance element is in contact with the contact pin 106 of the substrate 105, the head module recognition unit 15 may recognize that the head module 200 is mounted on the main body 110, and when it is detected that the resistance element is not in contact with the contact pin 106, the head module recognition unit 15 may recognize that the head module 200 is not mounted on the main body 110.

In addition, when the head module 200 is mounted on the main body 110, the control unit 11 may also recognize the type of the head module 200. The control unit 11 may calculate the resistance value of the resistance element provided in the head module 200 mounted on the main body 110 to recognize the type of the head module 200. Specifically, the head module recognition unit 15 may calculate the resistance value of the resistance element contacted through the contact pin 106 of the substrate 105 to recognize the type of the head module 200. For example, when the resistance value of the resistance element in contact with the contact pin 106 is a first value, the head module recognition unit 15 may recognize the head module 200 as the diagnostic module 210, when the resistance value of the resistance element in contact with the contact pin 106 is a second value, the head module recognition unit 15 may recognize the head module 200 as the skin care module 220, when the resistance value of the resistance element in contact with the contact pin 106 is a third value, the head module recognition unit 15 may recognize the head module 200 as the scalp care module 230, and when the resistance value of the resistance element in contact with the contact pin 106 is a fourth value, the head module recognition unit 15 may recognize the head module 200 as the ampoule care module 240.

The cosmetic device 1 may continuously detect whether the head module 200 is mounted in a state in which the head module 200 is not mounted.

When the cosmetic device 1 detects that the head module 200 is mounted, the cosmetic device 1 may transmit information on the head module 200 to the mobile terminal 3 (S13).

When the mounting of the head module 200 is detected, the control unit 11 may control the communication unit 17 to transmit the information on the head module 200 to the mobile terminal 3. The communication unit 17 may transmit the information on the recognized head module 200 to the mobile terminal 3.

The information on the head module 200 may refer to information related to the head module 200 currently mounted on the main body 110. For example, the information on the head module 200 may include information on the type of the head module 200, recognition information on the head module 200, and the like.

The information on the type of the head module 200 may be the resistance value of the resistance element provided in the head module 200 or may include a model name, a device name, and function information of the head module 200.

The recognition information on the head module 200 may include status information diagnosed by the diagnosis module 210, information on care result information performed by the care modules 220, 230, and 240, and the like.

The mobile terminal 3 may receive the information on the head module 200 from the cosmetic device 1. The control unit 31 of the mobile terminal 3 may receive the information on the head module 200 from the cosmetic device 1 through the communication unit 45.

The mobile terminal 3 may determine the method of operating the cosmetic device 1 (S15).

When the mobile terminal 3 receives the information on the head module 200 from the cosmetic device 1, the mobile terminal 3 may determine the method of operating the cosmetic device 1 based on the information of the head module 200.

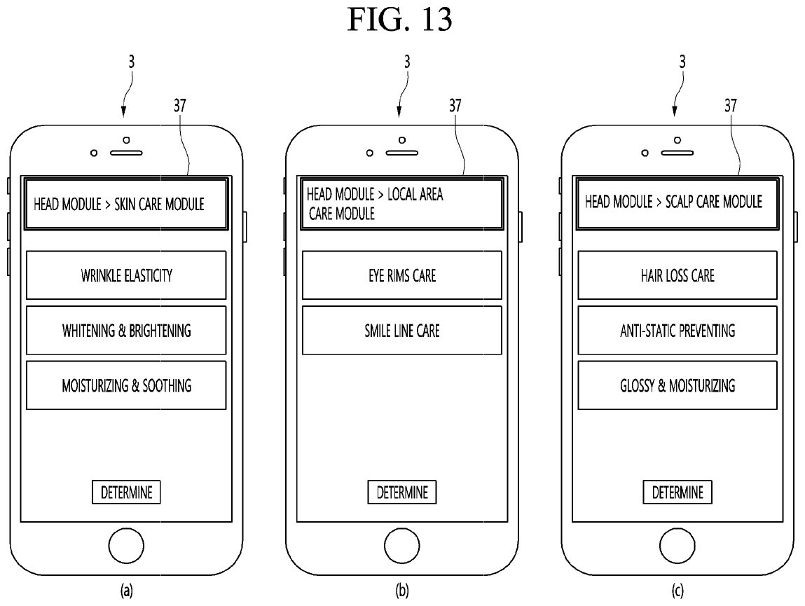

Next, a method for determining a method of operating a cosmetic device in a cosmetic device system according to an embodiment of the present disclosure will be described with reference to FIGS. 13 to 16.

FIG. 13 is a view illustrating a first example of a screen displayed by a mobile terminal to determine the method of operating the cosmetic device according to an embodiment of the present disclosure.

When the mobile terminal 3 receives the information on the head module 200, the mobile terminal 3 may control a display 37 so as to display an operation mode provided by the head module 200 based on the information on the head module 200.

For example, when the head module 200 is the general skin care module 220, the control unit 31 may control the display 37 so that operation modes (e.g., wrinkle elasticity mode, whitening & brightening mode, moisturizing & soothing mode, etc.) provided by the skin care module 220 are displayed as shown in FIG. 13A, when the head module 200 is the local area skin care module, the control unit 31 may control the display 37 so that operation modes (e.g., eye rims care mode, smile line care mode, etc.) provided by the local area skin care module mode are displayed as shown in FIG. 13B, and when the head module 200 is the scalp care module 230, the control unit 31 may control the display 37 so that operation modes (e.g., a hair loss care mode, an anti-static mode, a glossy & moisturizing mode, etc.) provided by the scalp care module 230 are displayed as shown in FIG. 13C.

That is, when the information on the head module 200 is different, the mobile terminal 3 may differently display the operation mode. As such, the mobile terminal 3 automatically displays functions provided by the head module 200 based on the information on the head module 200, and thus there is no need for the user to separately input which head module 200 is mounted on the cosmetic device 1, and there is an advantage that the function of the head module 200 may be easily guided.

When the control unit 31 receives a selection command of any one of the operation modes displayed on the display 37 as shown in FIG. 13, the control unit 31 may determine the selected operation mode as the method of operating the cosmetic device 1.

For example, the control unit 31 may determine any one of the wrinkle elasticity mode, the whitening & brightening mode, and the moisturizing & soothing mode as the method of operating the cosmetic device 1, and an operation method according to each operation mode may be as shown in Table 1 below, but this is merely illustrative, and thus the present disclosure is not limited thereto.

TABLE 1

| | Operating | Radio | | | |
|---|---|---|---|---|---|
| Mode | time | Frequency | Vibration | Current | LED |
| wrinkle | 3 mins | 1 MHz, 60~80 | Constant | | Red |
| elasticity | | V(Target temp | 2,000, 3000 or | | 630 +/− 20 nm |
| | | 39° C.)under | 4,000 rpm | | |
| | | 5 secs | | | |
| whitening & | 3 mins | X | Sine1,000~3,000 | 200 μA(Default), | Green |
| brightening | | | rpm | Direct Current, −Current, | 530 +/− 20 nm |
| | | | | 100~300 moditable | |
| | | | | by BT | |
| moisturizing & | 2 mins | X | Constant | 100 μA (Default), | Blue |
| soothing | | | 2,000, 3000 or | Plus Current, 50 Hz, | 470-20 nm |
| | | | 4,000 rpm | Duty 50%, 100 or 200 | |
| | | | | moditable by BT | |

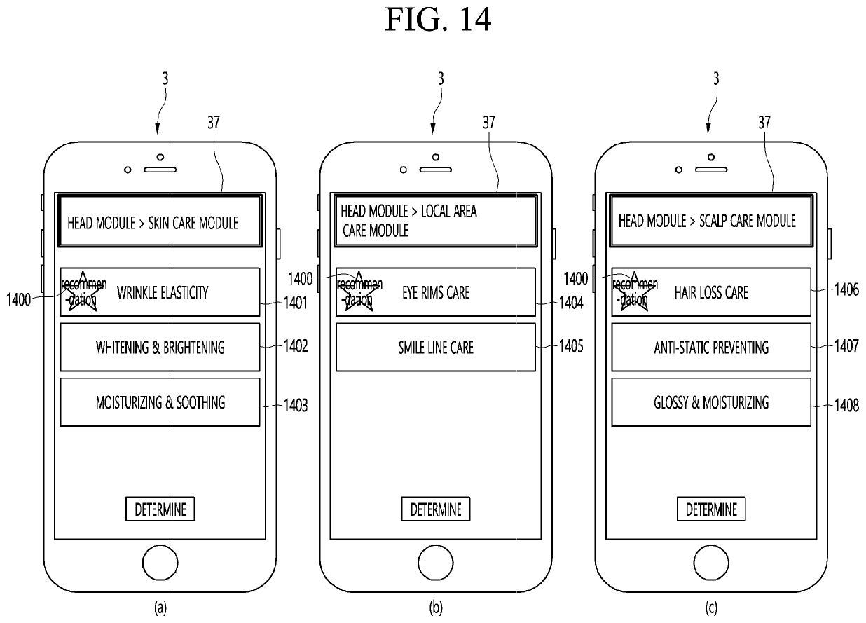

FIG. 14 is a view illustrating a second example of the screen displayed by the mobile terminal to determine the method of operating the cosmetic device according to an embodiment of the present disclosure. The mobile terminal 3 may receive information on the diagnosis module 210 and information on the care modules 220, 230, and 240 as the information on the head module 200. That is, the mobile terminal 3 may continuously receive the information on the diagnosis module 210 and the information on the care modules 220, 230, and 240. To this end, the user may mount the care modules 220, 230, and 240 on the main body 110 after the diagnosis module 210 is mounted on the main body 110 to diagnose the current state. Conversely, the user may mount the diagnosis module 210 on the main body 110 after the care modules 220, 230, and 240 are mounted on the main body 110.

When the mobile terminal 3 receives the information on the diagnosis module 210 and then receives the information on the care modules 220, 230, and 240, the display 37 may be controlled so as to display an icon that indicates an operation mode provided by the care modules 220, 230, and 240 and an operation mode recommended according to the diagnosis result of the diagnosis module 210 among the operation modes.

For example, after the control unit 31 receives the diagnosis result of the skin condition as the information on the diagnosis module 210, the control unit 31 may receive information on the skin care module 220. The control unit 31 may recognize, an operation mode related to a state having the lowest score in the skin condition diagnosis result among the operation modes provided by the skin care module 220 as a recommended operation mode. In an example of FIG. 14A, the control unit 31 may recognize that a score of the wrinkle state among wrinkles, whitening, and moisturizing is the lowest. In this case, as shown in FIG. 14A, the control unit 31 may display operation modes 1401, 1402, and 1403 provided by the skin care module 220 and may control the display 37 so that a recommendation icon 1400 is displayed in the operation mode recommended according to the diagnosis result among the displayed operation modes 1401, 1402, and 1403.

Meanwhile, the recommendation icon 1400 may be displayed in one or more operation modes. For example, when the score of the wrinkle state and the whitening state in the diagnosis result of the skin state is low, the control unit 31 may control so that the display 37 displays the recommendation icon 1400 in the two operation modes 1401 and 1402, unlike in FIG. 14A.

The control unit 31 may receive a command that selects two or more operation modes. When the control unit 31 receives the command that selects two or more operation modes, the control unit 31 may determine the method of operating the cosmetic device 1 so that the selected plurality of operation modes are sequentially performed.

When the mobile terminal 3 receives the information on the diagnosis module 210 and then receives the information on the care modules 220, 230, and 240, the mobile terminal 3 may determine the operation method so that a plurality of operation modes provided by the care module 220, 230, and 240 on the basis of the diagnosis result of the diagnosis module 210 are continuously performed.

As another example, after control unit 31 may receive the diagnosis result of the skin condition as the information on the diagnosis module 210, the control unit 31 may receive information on the local area skin care module. The control unit 31 may recognize an operation mode related to a state having the lowest score in the skin condition diagnosis result among operation modes provided by the local area skin care module as a recommended operation mode. In the example of FIG. 14B, the control unit 31 may recognize that a score of a state of the eye rims among the eye rims and the mouth area is the lowest. In this case, as shown in FIG. 14B, the control unit 31 may display operation modes 1404 and 1405 provided by the local area skin care module and may control the display 37 so that a recommendation icon 1400 is displayed in the operation mode recommended according to the diagnosis result among the displayed operation modes 1404 and 1405.

As still another example, the control unit 31 may receive the diagnosis result of a scalp condition as the information on the diagnosis module 210 and then may receive information on the scalp care module 230. The control unit 31 may recognize an operation mode related to a state having the lowest score in the diagnosis result of the scalp condition among operation modes provided by the scalp care module 230 as a recommended operation mode. In the example of FIG. 14C, the control unit 31 may recognize that a score of a hair loss state among hair loss, static electricity, and gloss is the lowest. In this case, as shown in FIG. 14C, the control unit 31 may display operation modes 1406, 1407, and 1408 provided by the scalp care module 230 and may control the display 37 so that a recommendation icon 1400 is displayed in the operation mode recommended according to the diagnosis result among the displayed operation modes 1406, 1407, and 1408.

As such, according to an example of FIG. 14, it is possible not only to guide the user to the operation modes provided by the care module currently mounted on the main body 110, but also to guide the operation mode recommended to the user among the operation modes, and thus it is possible to provide convenience to use the cosmetic device 1 to the user.

When the control unit 31 receives a command to select any one of the operation modes displayed on the display 37 as shown in FIG. 14, the control unit 31 may determine the selected operation mode as the method of operating the cosmetic device 1. According to the example of FIG. 14, the user may easily select the operation mode through the recommendation icon 1400.

Meanwhile, in the example of FIG. 14, the control unit 31 recommends any one of the operation modes of the care modules 220, 230, and 240, but according to an embodiment, the control unit 31 may generate a recommended operation mode separate from the operation modes of the care modules 220, 230, and 240 provided in advance.

Specifically, when the mobile terminal 3 receives the information on the diagnosis module 210 and then receives the information on the care modules 220, 230, 240, the mobile terminal 3 may generate an operation mode recommended based on a function provided by the care modules 220, 230, and 240 and the diagnostic result of the diagnostic module 210. For example, when the control unit 31 determines that wrinkle and whitening care is required based on the information on the diagnosis module 210 and receives the information on the skin care module 220 as the information on the head module 200, the control unit 31 may generate an operation mode for improving each of wrinkles and whitening through the skin care module 220. That is, the control unit 310 may generate a new operation mode in which a LED (not shown), a vibration generator (not shown), a high-frequency laser (not shown), etc. that are provided in the skin care module 220 are driven based on the information on the diagnosis module 210.

For example, when the head module 200 is the skin care module 220, the control unit 31 may generate an operation mode such as mode 1, mode 2, or mode 3 of Table 2 below, or when the head module 200 is the scalp care module 230, the control unit 31 may generate the operation mode such as mode 1, mode 2, or mode 3 of Table 2 below. The mode 1, mode 2, or mode 3 may be a combination of predetermined operation modes or a newly generated mode according to the diagnosis result.

TABLE 2

| Head | Mode | LED | Galvanic | Ion | Vibration |
|---|---|---|---|---|---|
| skin | mode 1 | red | 200 μA@ | | Vibrating medium |
| | | | 150 kΩ | | (70%) |
| | mode 2 | blue | 200 μA@ | | Vibrating strong |
| | | | 150 kΩ | | (100%) |
| | mode 3 | red and blue | 200 μA@ | | vibration |
| | | | 150 kΩ | | pattern |
| scalp | mode 1 | red | | 5 V | Vibrating medium |
| | | | | (10 Hz) | (70%) |
| | mode 2 | blue | | 5 V | Vibrating strong |
| | | | | (10 Hz) | (100%) |
| | mode 3 | red and blue | | 5 V | vibration |
| | | | | (10 Hz) | pattern |

As described in FIG. 14, when the care modules 220, 230, and 240 are mounted after the diagnosis module 210 is mounted on the main body 110, the control unit 11 of the cosmetic device 1 may control the care modules 220, 230, and 240 based on the diagnosis result of the module 210.

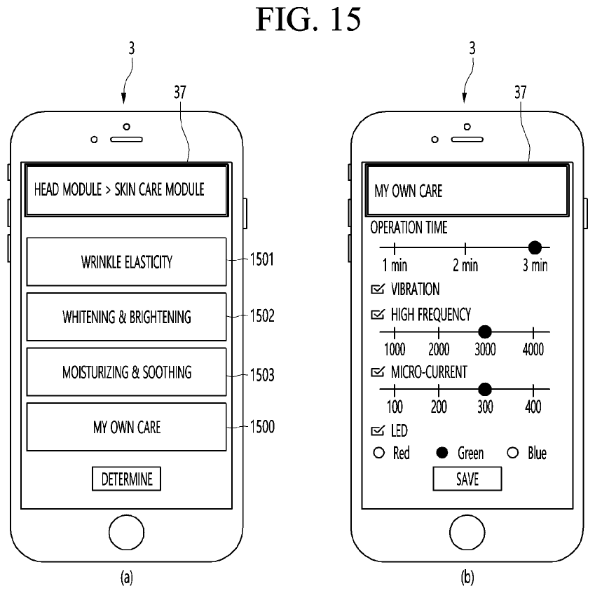

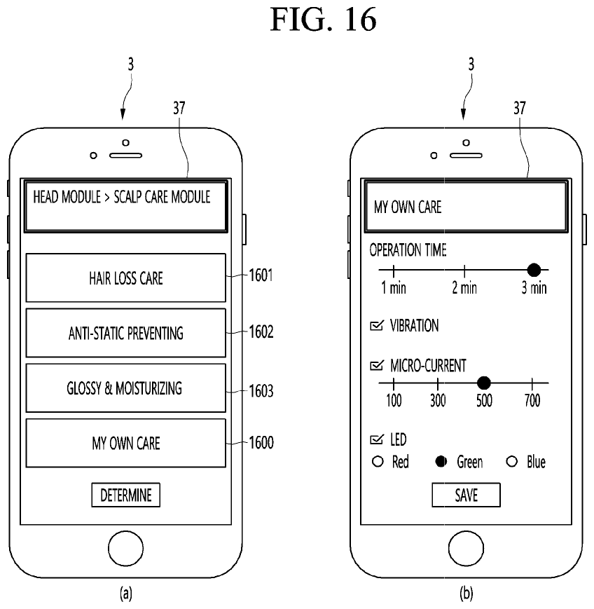

FIGS. 15 and 16 are views illustrating a third example of the screen displayed by the mobile terminal to determine the method of operating the cosmetic device according to an embodiment of the present disclosure.

The mobile terminal 3 may receive a user input that sets a method of operating the head module 200 and generate an operation mode corresponding to the user input.

Specifically, when the mobile terminal 3 receives the information on the head module 200, the mobile terminal 3 may control the display 37 so as to display an operation mode provided by the head module 200 and an operation mode creation icon. The operation mode creation icon is an icon for creating a customized operation mode and may be an icon for setting a method of operating the cosmetic device 1 preferred by an individual user.

For example, as shown in FIG. 15, when the control unit 31 receives the information on the skin care module 220, the control unit 31 may control the display 37 so as to display operation modes 1501, 1502, and 1503 provided by the skin care module 220 and an operation mode creation icon 1500. As another example, as shown in FIG. 16, when the control unit 31 receives the information on the scalp care module 230, the control unit 31 may control the display 37 so as to display operation modes 1601, 1602, and 1603 provided by the scalp care module 230 and an operation mode creation icon 1600.

When the control unit 31 receives a command to select the operation mode creation icons 1500 and 1600, the control unit 31 may control the display 37 so as to display an operation mode creation screen as shown in FIG. 15B or FIG. 16B.

The operation mode creation screen is a screen displayed when the operation mode creation icon is selected and may be displayed differently according to the information on the head module 200.

The mobile terminal 3 may differently display a method of operating the head module 200 that may be set according to the head module 200 mounted on the cosmetic device 1. The operation method may include an operation time, whether vibration or not and strength, whether or not to supply a micro-current and strength, and whether or not to supply a high frequency and strength.

When the skin care module 220 is mounted on the cosmetic device 1, the control unit 31 may display the operation mode creation screen so that the operation method is set within an operable range of the skin care module 220, and when the scalp care module 230 is mounted on the cosmetic device 1, the control unit 31 may display the operation mode creation screen so that the operation method is set within an operable range of the scalp care module 230.

For example, when the skin care module 220 is mounted in the cosmetic device 1, the control unit 31 may display an operation mode creation screen in which at least one of the operation time, whether the vibration or not, high-frequency intensity, micro-current intensity, and LED color may be set as shown in FIG. 15B, and when the skin care module 220 is mounted in the cosmetic device 1, the control unit 31 may control the display 37 so as to display the operation mode creation screen in which at least one of the operation time, whether the vibration or not, micro-current intensity, and LED color may be set as shown in FIG. 16. In addition, as shown in FIG. 15B or FIG. 16B. the control unit 31 may differently display a range of settable micro-current strength according to the head module 200 mounted on the cosmetic device 1. That is, when the skin care module 220 is mounted on the cosmetic device 1, the control unit 31 may display the operation mode creation screen so that the micro-current is set at 100 μA to 400 μA, but when the scalp care module 230 is mounted on the cosmetic device 1, the control unit 31 may control the display 37 so as to display the operation mode creation screen such that the micro current is set between 100 μA and 700 μA.

That is, the mobile terminal 3 may differently display the operation mode creation screen according to the head module 200 mounted on the cosmetic device 1.

Accordingly, the mobile terminal 3 displays the operation mode creation screen so that the operation mode of the cosmetic device 1 is generated within an operable range according to the head module 200, and thus there is an advantage that it is possible to prevent the operation mode from being generated so as to deviate from the performance of the head module 200 and to minimize an operation error of the head module 200.

When the control unit 31 receives a command to select any one of the operation modes displayed on the display 37 as shown in FIG. 15 or FIG. 16, the control unit 31 may determine the selected operation mode as the method of operating the cosmetic device 1. According to an example of FIG. 15 or FIG. 16, the mobile terminal 3 may determine the operation mode generated by the user as the method of operating the cosmetic device 1.

Again, FIG. 12 will be described.

The control unit 31 may transmit information on the determined operation method to the cosmetic device 1 (S17).

The control unit 11 of the cosmetic device 1 may operate according to the information received by the mobile terminal 3 (S19).

The control unit 11 may differently control the operation of the head module 200 mounted on the main body 110 according to the information received by the mobile terminal 3.

Even when the head module 200 mounted on the main body 110 is the same, the control unit 11 may differently control the operation of the head module 200 according to the information received by the mobile terminal 3. For example, when the skin care module 210 is mounted on the main body 110, the control unit 11 may differently control an operation of the skin care module 210 when the mobile terminal 3 receives the wrinkle elasticity mode as the operation method and an operation of the skin care module 210 when the mobile terminal 3 receives the whitening & brightening mode as the operation method. That is, even though the same head module 200 is mounted, the user may receive skin or scalp care through the mobile terminal 3 in an operation mode required by the user.

According to an embodiment of the present disclosure, the above-described method may be implemented as processor-readable code in a medium on which a program is recorded. Examples of the processor-readable medium include a ROM, a RAM, a CD-ROM, a magnetic tape, a floppy disk, an optical data storage, and the like.

The above-described descriptions are merely illustrative of the technological spirit of the present invention, and various modifications and variations may be made by those having ordinary skill in the art to which the present invention pertains without departing from the essential characteristics of the present invention.

Therefore, the embodiments disclosed in the present invention are not intended to limit the technological spirit of the present invention, but the embodiments are intended to describe, and the spirit and scope of the present invention is not limited by such embodiments.

The protection scope of the present invention should be construed by the following claims, and all technological spirits within the equivalent scope thereof should be construed as being included in the scope of right of the present invention.

1. A cosmetic device comprising:
a main body;
a head module detachable from the main body;
a control unit for recognizing the head module mounted on the main body; and
a communication unit for transmitting information on the recognized head module to a mobile terminal,
wherein the control unit differently controls an operation of the head module mounted on the main body according to the information received by the mobile terminal.

2. The cosmetic device of claim 1, wherein the control unit differently controls the operation of the head module according to the information received by the mobile terminal even when the head module mounted on the main body is the same.

3. The cosmetic device of claim 1, wherein when a care module is mounted after a diagnosis module is mounted on the main body, the control unit controls the care module based on a diagnosis result of the diagnosis module.

4. The cosmetic device of claim 1, wherein the head module includes at least one of a diagnosis module, a skin care module, a scalp care module, and an ampoule care module.

5. The cosmetic device of claim 4, wherein each of the diagnosis module, the skin care module, the scalp care module, and the ampoule care module include a resistance element having a different resistance value, and
the control unit calculates the resistance value of the resistance element provided in the head module mounted on the main body to recognize the head module.

6. The cosmetic device of claim 4, wherein a space in which a cartridge is mounted and a hole through which an ampoule accommodated in the cartridge passes are formed in the ampoule care module.

7. A cosmetic device system comprising:
a cosmetic device with a detachable head module; and
a mobile terminal that determines a method of operating the cosmetic device based on information on the head module mounted on the cosmetic device and transmits information on the determined operation method to the cosmetic device,
wherein the cosmetic device differently controls an operation of the head module according to the information received by the mobile terminal.

8. The cosmetic device system of claim 7, wherein the cosmetic device differently controls the operation of the head module according to the information received by the mobile terminal even when the mounted head module is the same.

9. The cosmetic device system of claim 7, wherein the mobile terminal displays an operation mode provided by the head module based on the information on the head module.

10. The cosmetic device system of claim 9, wherein the mobile terminal differently displays the operation mode when the information on the head module is different.

11. The cosmetic device system of claim 9, wherein when the mobile terminal receives information on a diagnostic module and then receives information on a care module, the mobile terminal displays an icon that indicates an operation mode provided by the care module and an operation mode recommended according to a diagnosis result of the diagnostic module among the operation modes.

12. The cosmetic device system of claim 9, wherein when the mobile terminal receives the information on the diagnosis module and then receives the information on the care module, the mobile terminal generates an operation mode recommended based on a function provided by the care module and the diagnosis result of the diagnosis module.

13. The cosmetic device system of claim 9, wherein when the mobile terminal receives the information on the diagnostic module and then receives the information on the care module, the mobile terminal determines an operation method so that a plurality of operation modes provided by the care module are continuously executed based on the diagnostic result of the diagnostic module.

14. The cosmetic device system of claim 7, wherein the mobile terminal receives a user input for setting a method of operating the head module and generates an operation mode corresponding to the user input.

15. The cosmetic device system of claim 14, wherein the mobile terminal differently displays a settable method of operating the head module according to the head module mounted on the cosmetic device.
